(19) United States
(12) Patent Application Publication
(10) Pub. No.: US 2016/0071408 A1
(43) Pub. Date: Mar. 10, 2016

(54) WEARABLE APPARATUS AND DATA PROCESSING METHOD

(71) Applicants: Beijing Lenovo Software Ltd., Beijing (CN); Lenovo (Beijing) Limited, Beijing (CN)

(72) Inventors: Jing'en Jiao, Beijing (CN); Tao Miao, Beijing (CN); Jixun Zhao, Beijing (CN)

(21) Appl. No.: 14/640,258

(22) Filed: Mar. 6, 2015

(30) Foreign Application Priority Data
Sep. 9, 2014 (CN) 201410455802.1
Dec. 29, 2014 (CN) 201410849275.2

(51) Int. Cl. G08C 17/02; A61B 5/00

(52) U.S. Cl. CPC G08C 17/02; A61B 5/6802

(57) ABSTRACT

The present disclosure provides a wearable apparatus and a data process method. The wearable apparatus includes a first body and a control unit, the first body has different configurations to be worn by a user, the control unit is configured to acquire a first parameter as the wearable apparatus is worn by the user, and generate a control instruction based on the first parameter to adjust the first body to one of the different configurations.

### CROSS-REFERENCE TO RELATED APPLICATION

This application claims the benefit of priority to Chinese Patent Applications No. 201410455802.1 filed on Sep. 9, 2014, and to Chinese Patent Application No. 201410849275.2 filed on Dec. 29, 2014, the disclosures of which are incorporated herein by reference in their entirety.

### FIELD

The present disclosure generally relates to a technical field of electronics, in particular to a wearable apparatus and a data processing method.

### BACKGROUND

A wearable (electronic) apparatus such as a smart watch or a smart bracelet becomes popular due to its portability and smartness. Conventionally, the tension of the wearable apparatus needs to be adjusted manually when the user wears it, and thus the user's experience is poor.

The international computer academia and industry all along pay attention to the wearable technology. However, due to high cost and complicated technology, many relative apparatuses only are still in idea. As the advance of technology, in particular, the development of the mobile network and the appearance of high performance and low power consumption process chips, a part of the wearable apparatuses have been commercialized from conceptualization and novel wearable apparatuses have been continuously proposed and many technical companies also begin to research deeply in the new field.

The wearable apparatus is a portable apparatus which is worn directly on the body of the user, or is integrated in the clothes or accessories of the user. The wearable apparatus is not only a hardware apparatus, but also achieves a strong function by support of software, data interactions and cloud interactions. The wearable apparatus will change our lives and acknowledge greatly.

Typically, the people may have different requirements to the configurations of the wearable apparatus when they are in different states. However, the conventional wearable apparatus has a fixed configuration, or only may be adjusted manually by the user, instead of being adjusted automatically to meet different requirements of the user in different states to the configurations.

### SUMMARY

In accordance with an aspect of the present disclosure, a wearable apparatus is provided, which includes: a first body having different configurations; and a control unit configured to acquire a first parameter and generate a control instruction based on the first parameter to adjust the first body to one of said different configurations.

In an example, the control unit includes: a first detection unit configured to detect the first parameter; a first analyzing unit configured to analyze the first parameter to acquire a first analyzing result; and a first command unit configured to generate the control instruction from the first analyzing result to adjust the first body to one of said different configurations based on the acquired first parameter.

In an example, the control unit further includes a sensor configured to acquire the first parameter, and the sensor is one of a pressure sensor, a gravity sensor, an acceleration sensor, and a sensor for detecting physiological data of a human body.

In an example, the first body includes at least a first device and a first deformable body; the first device is operable to deform the deformable body.

In an example, the wearable apparatus is a smart watch, the first device is a gas pump, the first deformable body is a gasbag in a watchband of the smart watch, and a tension of the watchband is adjustable by the gas pump.

In an example, the gasbag includes an electrolyte and the tension of the watchband is further adjustable by heating or cooling the electrolyte, or a combination of both.

In an example, the wearable apparatus is a smart watch, the first body includes a plurality of sub-watchbands constituting a watchband of the smart watch, each of the sub-watchbands includes a micro-motor and a plurality of fasteners, and a length of each of the sub-watchbands is adjustable depending on an engagement between a respective one of the micro-motors and a corresponding one of the fasteners. In an example, the first body further includes a first adjusting unit operable to cause the micro-motor to produce a first force, and at least one of the sub-watchbands is operable to produce a first displacement under the first force to adjust the first body to have a first length; or the first adjusting unit is operable to cause the micro-motor to produce a second force, and at least one of the sub-watchbands is operable to produce a second displacement under the second force to adjust the first body to have a second length, the second length is different from the first length.

In an example, the micro-motor is connected with the fasteners by an elastic member.

In an example, the wearable apparatus further includes a second body, the first body and the second body are combined to form the whole frame of the wearable apparatus.

In an example, the first body includes a first assembly formed by a deformable material and a force transmission assembly, and the force transmission assembly is operable to change a force applied to the first assembly to adjust the first assembly to one of said different configurations based on the acquired first parameter.

In an example, the force transmission assembly is a controllable reel.

In an example, the first body includes a gas deflation and aeration assembly, and a gas pressure assembly; the first body is operable to adjust an amount of gas entering the gas deflation and aeration assembly based on the control instruction to adjust the first body to one of said different configurations based on the acquired first parameter. In an example, the wearable apparatus further includes a gas cushion buffer assembly for contacting with a human body.

In an example, the wearable apparatus further includes:

a sensor configured to detect an environmental space parameter in a predetermined range, the sensor includes one or more of a pressure sensor, a temperature sensor and a humidity sensor and any combination thereof, and

the control unit is further configured to receive the environmental space parameter and to generate the control instruction based on the received environmental space parameter.

In an example, the control unit is further configured to generate the control instruction according to habits or custom of the user who uses the wearable apparatus.

In an example, the control unit is configured to generate the control instruction based on a control command transmitted by the user.

In an example, the control unit is further configured to do timekeeping after the control instruction is transmitted; and if the duration for which timekeeping is done is greater than a time threshold, the control unit is operable to generate an alarm information for prompting the user to adjust the first body to one of the different configurations.

In accordance with another aspect of the present disclosure, it provides a data processing method configured to process data in a wearable apparatus which includes a first body having different configurations and a control unit configured to generate a control instruction based on a received first parameter to adjust the first body to one of the different configurations, the method includes: detecting the first parameter; analyzing the first parameter to acquire a first analyzing result; and adjusting the first body to one of the different configurations based on the first analysing result.

In accordance with a further aspect of the present disclosure, it provides a data processing method for processing data in a wearable apparatus which includes a first body and a control unit, the first body has different configurations, the method includes: generating a control instruction by the control unit based on a received parameter information; transmitting the control instruction to the first body; and adjusting the first body to one of the different configurations based on the control instruction.

### BRIEF DESCRIPTION OF THE DRAWINGS

FIG. 1 is a schematic flow chart of a data process method according to a first embodiment of the present disclosure;

FIG. 2 is a schematic flow chart of steps further incorporated in FIG. 1;

FIG. 3 is another schematic flow chart of steps further incorporated in FIG. 1;

FIG. 4 is a schematic cross-sectional view of a watchband as an example to explain the flow chart shown in FIG. 2;

FIG. 5(a)-(c) are schematic views of another watchband as an example to explain the flow chart shown in FIG. 3;

FIG. 6 is a schematic view for showing a structure of the wearable apparatus provided by a first embodiment of the present disclosure;

FIG. 7 is a schematic view for showing a first structure of the wearable apparatus of a second embodiment of the present disclosure;

FIG. 8 is a schematic view of a structure of a reel of the wearable apparatus shown in FIG. 7;

FIG. 9 is a schematic view for showing a second structure of the wearable apparatus of the second embodiment of the present disclosure;

FIG. 10 is a schematic view for showing a third structure of the wearable apparatus of the second embodiment of the present disclosure;

FIG. 11 is a schematic view for showing a fourth structure of the wearable apparatus of the second embodiment of the present disclosure;

FIG. 12 is a schematic view for showing a fifth structure of the wearable apparatus of the second embodiment of the present disclosure; and

FIG. 13 is a schematic flow chart of a data process method according to the second embodiment of the present disclosure.

### DETAILED DESCRIPTION OF THE EMBODIMENTS

Embodiments of the present disclosure will be described hereinafter in more detail with reference to figures of the attached drawings. It should be noted that the embodiments are only used to explain the present disclosure by way of examples, rather than being construed as a limiting to the present invention.

### First Embodiment

In the following examples of the data process method (or information process method) and the wearable apparatus provided by the first embodiment of the present disclosure, the referred electronic apparatus includes, but not limited to, a smart watch, a smart bracelet, smart glasses, and the like. The preferred electronic apparatus in the first embodiment of the present disclosure is the smart watch.

A first example of the present disclosure provides an information process method applied to a wearable electronic apparatus. The wearable apparatus includes at least a first body configured to be capable of being in a first configuration or in a second configuration. The wearable apparatus is preferably the smart watch. The first body is preferably a watchband of the smart watch. The watchband in this example has adjustable tension. In the first configuration, the watchband is in loose state. In the second configuration, the watchband is in tight state. Certainly, alternatively, in the first configuration, the watchband may be in tight state, and in the second configuration, the watchband may be in loose state. In the following examples of the present disclosure, it will be explained with reference to the example that the first configuration corresponds to the loose state while the second configuration corresponds to the tight state.

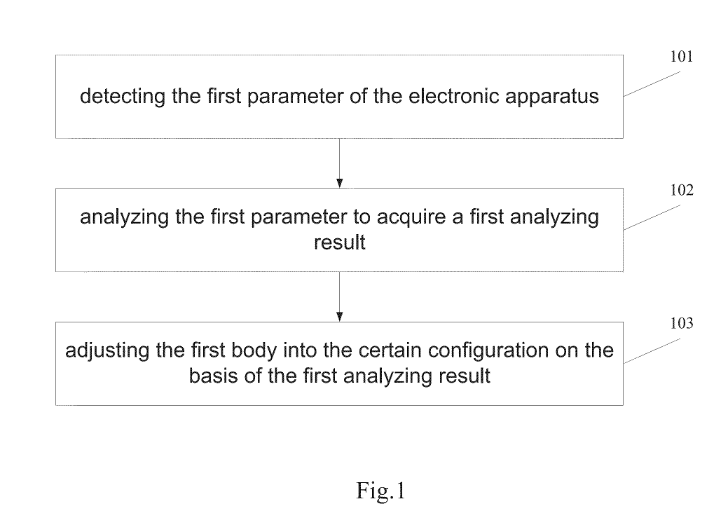

FIG. 1 is a schematic flow chart of a data process method according to a first embodiment of the present disclosure. As illustrated in FIG. 1, the method includes:

Step 101: detecting the first parameter of the electronic apparatus.

Herein, the first parameter may in particular be wearing tension of the smart watch. The tension of the watchband is adjusted on the basis of it. Further, when the user is in a mobile or immobile state, the state of the smart watch which is worn on a certain location, such as wrist, will become in the mobile or immobile state. Thus, on the basis of the speed or acceleration of the electronic apparatus, it may determine whether the smart watch is currently in the mobile state or in the immobile state. The first parameter is the speed or acceleration of the smart watch. In addition, considering that the physiological data such as heart rate, blood pressure or pulse, of the user will become different greatly in the two states when the user is in the mobile state or in the immobile state, the smart watch may determine whether the smart watch is in the mobile state or in the immobile state currently by detecting the physiological state of the user. At that time, the first parameter may in particular be physiological data for the user.

Step 102: analyzing the first parameter to acquire a first analyzing result.

Herein, from the first parameter detected by the smart watch, it may determine whether the smart watch is in a state to be loosened or in a state to be tightened.

Step 103: adjusting the first body into a certain configuration on the basis of the first analyzing result.

Herein, when it detects the smart watch worn currently is relatively tight, the smart watch will be adjusted to be loosened; when it detects the smart watch worn currently is relatively loose, the smart watch will be adjusted to be tightened. Considering that the user needs the smart watch he wears to be tighter in the mobile state and to be looser in the immobile state, for example a rest state, the watchband of the smart watch is adjusted to be tighter when the user is in the mobile state and to be looser when the user is in the immobile state.

In an example, the electronic apparatus further includes a first sensor which is a pressure sensor. The electronic apparatus may be worn on a first location of the user, such as the wrist of the user. The method further includes:

detecting a force of the electronic apparatus by the first sensor when the electronic apparatus is worn on the first location; determining whether the detected force is within a first predetermined range or not and generating a first determination result; generating a first instruction when the first determination result indicates the detected force is within the first predetermined range, and controlling the first body into the first configuration in response to the first instruction; generating a second instruction when the first determination result indicates the detected force is not within the first predetermined range, and controlling the first body into the second configuration in response to the second instruction.

In the above solution, the pressure sensor detects a pressure value between the wrist and the smart watch to acquire the wearing tension by detecting the pressure value. Since the user who wears one smart watch is relatively fixed and his wrist has a relatively fixed size in a certain period, the pressure value of the smart watch at the wrist is recorded in advance when the user feels it is worn tightly, so as to obtain a first predetermined pressure range; and/or the pressure value of the smart watch at the wrist is recorded in advance when the user feels it is worn loosely, so as to obtain a second predetermined pressure range. With reference to the example that only the first predetermined pressure range is recorded and the first configuration corresponds to the loose state of the watchband, when the smart watch detects the current pressure value by the pressure sensor, it determines whether the current pressure value detected is in the first predetermined pressure range or not; if yes, it indicates that the smart watch is currently worn too tightly and needs to be loosened, otherwise, the smart watch needs to be tightened.

In another example, the electronic apparatus includes a second sensor which is a gravity sensor or an acceleration sensor. The method further includes:

detecting a first movement parameter of the electronic apparatus by the second sensor; acquiring the variation of the first movement parameter within a first predetermined time; determining whether the variation meets a first variation condition or not and generating a second determination result; generating a first instruction when the second determination result indicates the variation meets the first variation condition, and controlling the first body into the first configuration in response to the first instruction; generating a second instruction when the second determination result indicates the variation does not meet the first variation condition, and controlling the first body into the second configuration in response to the second instruction.

In the above solution, when the second sensor is the gravity sensor, the first movement parameter is the speed; and when the second sensor is the acceleration sensor, the first movement parameter is the acceleration. The gravity sensor detects the speed of the smart watch or the acceleration sensor detects the acceleration of the smart watch. Since the variation of the speed or the acceleration of the user who wears one smart watch and the user himself/herself have certain characteristics within a certain period when the user is in mobile state or in immobile state, the variation range of the speed or acceleration of the smart watch is recorded in advance when the user is in the immobile state within a certain period such as 30 minutes and the recorded variation range is regarded as the first variation condition. With reference to the example that the second sensor is the acceleration sensor and the first configuration corresponds to the loose state of the watchband, the acceleration of the smart watch is detected by the acceleration sensor one time per 10 minutes within the 30 minutes and the variation rate of the acceleration of the smart watch within the 30 minutes is calculated. It determines whether the variation rate meets the first variation condition, that is, is in the variation range recorded in advance or not; if yes, it indicates that the user is in the immobile state within the 30 minutes and the first adjusting instruction will be generated to loosen the watchband, otherwise, it indicates that the user is in the mobile state within the 30 minutes and the second adjusting instruction will be generated to tight the watchband. The method for acquiring the speed by the gravity sensor can be found in the relevant prior art. The details will be omitted herein.

In a further example, the electronic apparatus includes a third sensor which may be a sensor for detecting the physiological data such as heart rate, blood pressure or pulse, of the user, in particular, may be a resonance sensor. The electronic apparatus may be worn on a first location of the user, such as the wrist of the user. The method further includes:

acquiring first data by the third sensor, the first data representing the physiological data of the user; determining whether the first data goes beyond a first preset range within a second preset period or not and generating a third determination result; generating a first instruction when the third determination result indicates the first data does not go beyond the first preset range within the second preset period, and controlling the first body into the first configuration in response to the first instruction; generating a second instruction when the third determination result indicates the first data goes beyond the first preset range within the second preset period, and controlling the first body into the second configuration in response to the second instruction.

In the above solution, the physiological data such as heart rate, blood pressure or pulse, of the user is detected by the resonance sensor. As the heart rate, blood pressure, pulse of the user who wears one smart watch may change significantly when the user is in mobile state or in immobile state, the variation of the physiological data of the user is recorded in advance when the user is in the immobile state and the recorded variation of the physiological data is regarded as the first preset range. With reference to the example that the resonance sensor detects the blood pressure and the first configuration corresponds to the loose state of the watchband, the blood pressure of the user is detected by the resonance sensor. It determines whether the detected blood pressure goes beyond the first preset range, that is, is in the variation range recorded in advance within a certain period, such as 6 minutes, or not; if yes, it indicates that the user is in the immobile state and the first adjusting instruction will be generated to loosen the watchband, otherwise, it indicates that the user is in the mobile state and the second adjusting instruction will be generated to tighten the watchband.

Thus, in the embodiments of the present disclosure, it may determine whether the watchband of the smart watch should be loosened or tightened by means of the analyzing result of the detected first parameter of the smart watch. It can adjust the tension of the wearable apparatus automatically to improve the user's experience, which can exhibit the diversity of function of an electronic apparatus.

On the basis of the above method, the response to the first instruction and the response to the second instruction will further be described below.

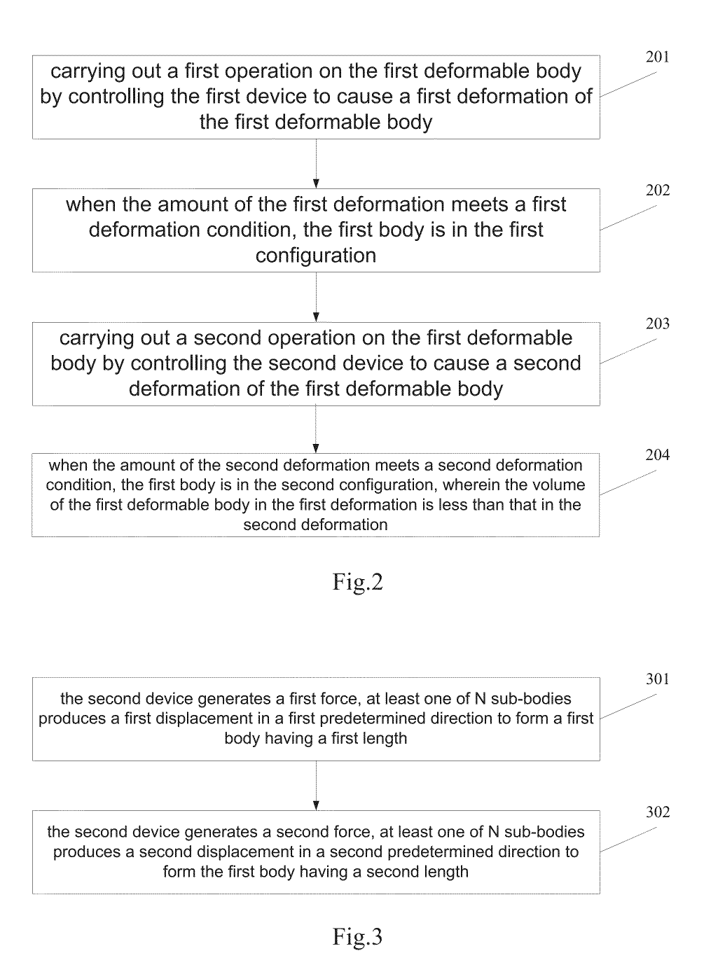

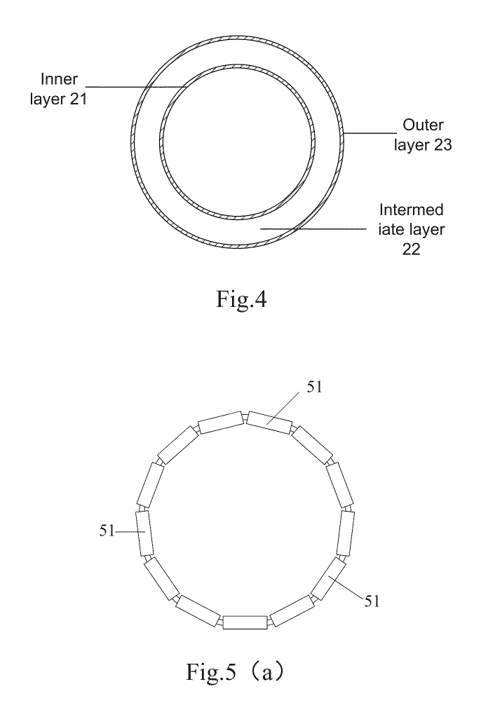

With reference to the watchband shown in FIG. 4, the respective steps in FIG. 2 are explained. The watchband is made of an entire gas bag, and includes an inner layer 21, an intermediate layer 22 and an outer layer 23. The inner layer 21 is inflatable and the outer layer 23 may be inflatable or may not be inflatable. The intermediate layer 22 is a hollow layer.

The electronic apparatus further comprises a first device and the first body at least comprises a first deformable body. The first body is the part other than the control unit or controller.

The method further includes:

Step 201: carrying out a first operation on the first deformable body by the first device to cause a first deformation of the first deformable body.

In an embodiment, the watchband of the smart watch is a soft watchband, for example, is made from rubber or soft leather. The first deformable body may be the gas bag. The soft watchband may include an entire gas bag, or may include a plurality of gas bags. They are not limited herein. The first device may be a gas pump, preferably, may be configured in the body of the smart watch (the part of the smart watch other than the watchband), or may be arranged in the watchband.

With reference to the example that the first configuration corresponds to the loose state of the watchband (i.e., it determines the user is in the immobile state currently and the watch is worn tightly and needs to be loosened), when the smart watch detects the operation of the user to a first predetermined key, the first predetermined key is used to deflate the watchband. In response to this operation, the gas pump performs the deflation operation to the intermediate layer 22. During the deflation, the volume of the intermediate layer 22 is reduced gradually such that the volume of the watchband becomes smaller. As the intermediate layer 22 is deflated continuously, the inner layer 21 reduces the tension to the wrist, i.e., reduces the pressure value of the watchband at the wrist such that the smart watch becomes more loose.

Step 202: when the amount of the first deformation meets a first deformation condition, the first body is in the first configuration.

Herein, the amount of released gas of the intermediate layer 22 is recorded in advance when the wrist of the user feels good; in the course of deflating the intermediate layer 22 of the soft watchband, when the amount of released gas is determined to reach the recorded value, the gas pump is forbidden to continue deflating the intermediate layer 22 to stop the deflation operation such that the user has a good comfort.

Step 203: carrying out a second operation on the first deformable body by the first device to cause a second deformation of the first deformable body.

Herein, as illustrated in FIG. 4, with reference to the example that the second configuration corresponds to the tight state of the watchband (i.e., it determines the user is in the mobile state currently and the watch is worn loosely and needs to be tightened), when the smart watch detects the operation of the user to a second predetermined key, the second predetermined key is used to inflate the watchband. In response to this operation, the gas pump performs the inflation operation to the intermediate layer 22. During the inflation, the volume of the intermediate layer 22 is increased gradually such that the volume of the watchband becomes larger. As the intermediate layer 22 is inflated continuously, the inflatable inner layer 21 expands to press the wrist tightly to increase the pressure value of the watchband at the wrist such that the smart watch becomes tighter.

Step 204: when the amount of the second deformation meets a second deformation condition, the first body is in the second configuration, wherein the volume of the first deformable body in the first deformation is smaller than the volume of it in the second deformation.

Herein, the amount of filling gas of the intermediate layer 22 is recorded in advance when the wrist of the user feels good; in the course of inflating the intermediate layer 22 of the soft watchband, when the amount of filling gas is determined to reach the recorded value, the gas pump is forbidden to continue inflating the intermediate layer 22 to stop the inflation operation such that the user has a good comfort.

In the above solution, it is explained with reference to the example that the intermediate layer 22 is deflated and inflated by the gas pump to achieve the adjusting of wearing tension of the watch. In addition, the intermediate layer 22 may also carry certain electrolyte which may be heated to cause the expanding of the intermediate layer 22. The electrolyte may be cooled to cause the intermediate layer 22 to contract. That is, the tension of the watchband to the wrist may be increased or reduced by heating or cooling the electrolyte. In particular, with reference to the example that the second configuration corresponds to the tight state of the watchband (i.e., it determines that the user is in the mobile state currently and the watch is worn loosely and needs to be tightened), when the smart watch detects the user operates a third predetermined key, the third predetermined key is used to heat the electrolyte in the intermediate layer 22 of the watchband. In response to the operation, the electrolyte is heated to cause the expanding of the intermediate layer 22. Thus, the pressure value between the watch and the wrist is increased. The heating time of the electrolyte is recorded in advance when the wrist of the user feels good. In the course of heating the electrolyte in the intermediate layer 22, when the heating time is determined to reach the recorded value, the operation of continuously heating the electrolyte will be forbidden, i.e., stop the heating such that the user has a good comfort.

As discussed above, in the example, the object of adjusting the wearing tension of the watch may be achieved by inflating and deflating the intermediate layer by the gas pump and/or by heating and cooling the electrolyte in the intermediate layer. It can adjust the tension of the wearable apparatus automatically to improve the user's experience, which can exhibit the diversity of function of an electronic apparatus.

On the basis of the above method shown in FIG. 1, the response to the first instruction and the response to the second instruction will further be described below.

The electronic apparatus further includes the second device. The first body at least includes N sub-bodies, where N is a positive integer.

The watchband provided by the present embodiment is a metal watchband. The sub-bodies are sub-watchbands, that is, the watchband includes N sections of sub-bodies. The second device may be a motor, in particular a micro-motor, which may be arranged in each of the sub-watchbands. Each of the sub-watchbands may include a first portion and a second portion. The lengths of the sub-watchbands may be increased or reduced by adjusting the tension of fasteners of the first portion and the second portion so as to increase or reduce the length of the entire watchband and achieve the adjusting of the tension.

When the first body is in the first configuration, the first body has a first length. When the first body is in the second configuration, the first body has a second length. The first length is greater than the second length. That is, the first configuration corresponds to the loose state and the second configuration corresponds to the tight state. The length of the watchband in the loose state is greater than that in the tight state.

The method further includes:

Step 301: the second device generates a first force, and at least one of the N sub-bodies produces a first displacement in a first predetermined direction under the first force to form the first body having a first length.

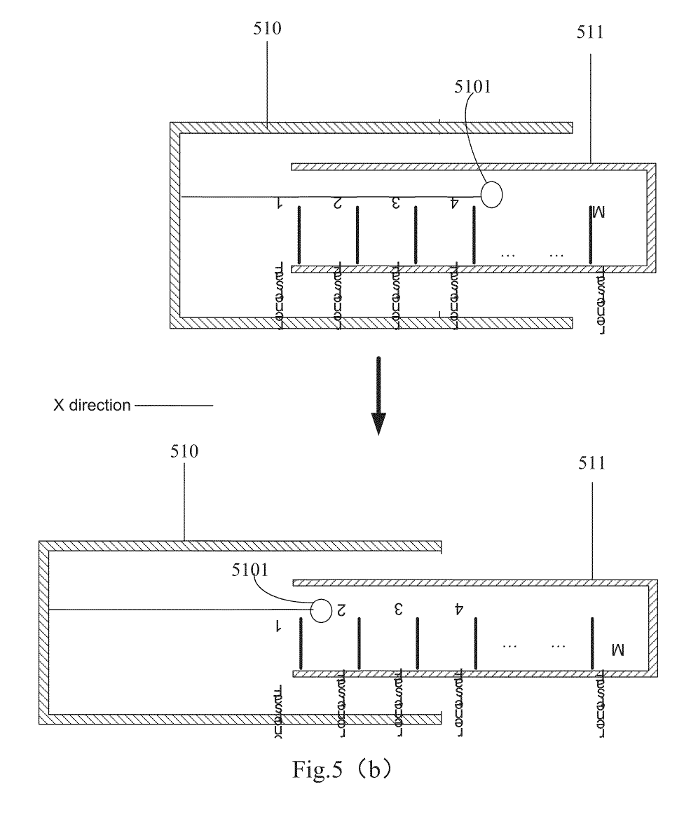

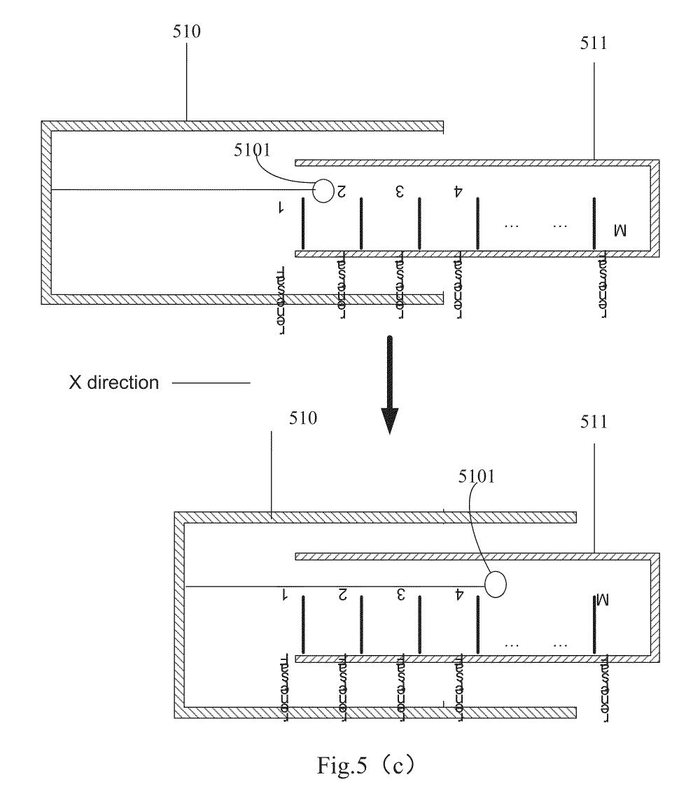

With reference to the example of the watchband shown in FIGS. 5(a)-(c), the respective steps in FIG. 3 will be explained below. In FIG. 5(a), the watchband is a metal watchband, which is formed by N sub-watchbands 51 connected together. In particular, FIGS. 5(b)-5(c) are cross-sectional views of one sub-watchband in different states. In FIGS. 5(b)-5(c), the first portion 510 of each of the sub-watchbands 51 includes a micro-motor 5101 and the second portion 511 includes M fasteners (fastener 1, fastener 2, . . . , fastener M), where M is a positive integer.

In the embodiment, it is defined as follows: the length of the sub-watchband formed by engaging the first portion 510 with the fastener 1 of the second portion 511 is greater than the length of the sub-watchband formed by engaging the first portion 510 with the fastener 2 of the second portion 511, and the length of the sub-watchband formed by engaging the first portion 510 with the fastener 2 of the second portion 511 is greater than the length of the sub-watchband formed by engaging the first portion 510 with the fastener 3 of the second portion 511, and do on.

Herein, with reference to the example that the first configuration corresponds to the loose state of the watchband (i.e., it determines the user is in the immobile state currently and the watch is worn tightly, for example, the first portion 510 is engaged with the fastener 4 of the second portion 511, and needs to be loosened), when the smart watch detects the operation of the user to a fourth predetermined key which is used to extend the sub-watchband, the micro-motor 5101 produces the first force in response to this operation and under the first force, as shown in FIG. 5(b), the first portion 510 that was previously engaged with the fastener 4 of the second portion 511 is engaged with the fastener 1 such that the engagement of the first portion 510 and the second portion 511 becomes looser and the sub-watchband becomes longer in the x axis to lengthen the entire watchband, that is, it obtains the longer watchband to achieve adjusting of the wearing tension.

Step 302: the second device produces a second force, and at least one of the N sub-bodies produces a second displacement in a second predetermined direction under the second force to form the second body having a second length.

Herein, with reference to the example that the second configuration corresponds to the tight state of the watchband (i.e., it determines the user is in the mobile state currently and the watch is worn loosely, for example, the first portion 510 is engaged with the fastener 1 of the second portion 511, and needs to be tightened), when the smart watch detects the operation of the user to a fifth predetermined key which is used to shorten the sub-watchband, the micro-motor 5101 produces the second force in response to this operation and under the second force, as shown in FIG. 5(c), the first portion 510 that was previously engaged with the fastener 1 of the second portion 511 is engaged with the fastener 4 such that the engagement of the first portion 510 and the second portion 511 becomes tighter and the sub-watchband becomes shorter in the x axis to shorten the entire watchband, that is, it obtains the shorter watchband to achieve adjusting of the wearing tension.

In the above solution, the variation of the length of the sub-watchband is achieved by varying the fastener of the second portion engaged with the first portion. Further, the first portion and the second portion may also be connected by springs, elastic connecting portions such as elastic strings to lengthen or shorten the sub-watchband by increasing or reducing the elasticity of these connecting portions and thus achieves the adjusting of wearing tension.

As discussed above, in the embodiment of the present disclosure, the sub-watchband may be lengthened or shortened to lengthen or shorten the entire watchband. It can adjust the tension of the wearable apparatus automatically to improve the user's experience, which can exhibits the diversity of function of an electronic apparatus.

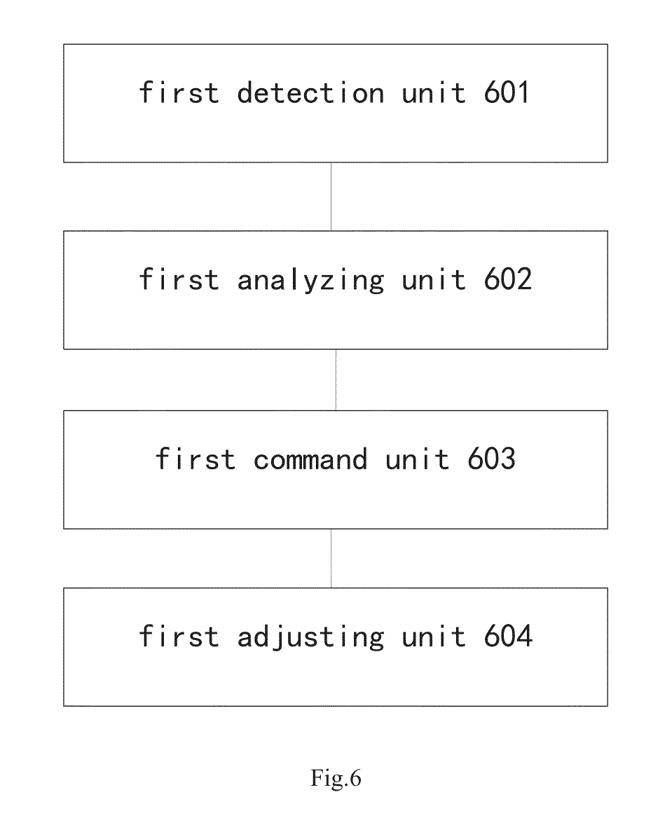

FIG. 6 is a schematic view for showing a structure of the wearable apparatus provided by a first embodiment of the present disclosure. As illustrated in FIG. 6, the electronic apparatus includes a control unit and a first adjusting unit 604. The control unit includes a first detection unit 601, a first analyzing unit 602 and a first command unit 603. It should be appreciated that the control unit and the first adjusting unit 604 may constitute one or more processors.

The first detection unit 601 is configured to detect the first parameter of the electronic apparatus.

Herein, the first parameter may in particular be wearing tension of the smart watch. The tension of the watchband is adjusted on the basis of it. Further, when the user is in a mobile or immobile state, the state of the smart watch which is worn on a certain location, such as wrist of the user, will become in the mobile or immobile state. Thus, the first detection unit 601 may determine whether the smart watch is currently in the mobile state or in the immobile state, by detecting the speed or acceleration of the electronic apparatus. The first parameter is the speed or acceleration of the smart watch. In addition, considering that the physiological data such as heart rate, blood pressure or pulse, of the user will become different greatly in the two states when the user is in the mobile state or in the immobile state, the first detection unit 601 may determine whether the smart watch is in the mobile state or in the immobile state currently by detecting the physiological state of the user. The first parameter may in particular be physiological data for the user.

The first analyzing unit 602 is configured to analyze the first parameter to acquire a first analyzing result.

Herein, from the first parameter detected by the smart watch, the first analyzing unit 602 may determine whether the smart watch is in a state to be loosened or in a state to be tightened.

The first command unit 603 is configured to generate a control command to adjust the first body into a certain configuration and/or needs different forces for deformation, on the basis of the first analyzing result.

The first adjusting unit 604 is configured to adjust the first body into the certain configuration on the basis of the control command.

Herein, when the first analyzing result indicates the smart watch worn currently is relatively tight, the first adjusting unit 604 will adjust the smart watch to be loosened; when the first analyzing result indicates the smart watch worn currently is relatively loose, the first adjusting unit 604 will adjust the smart watch to be tightened. Considering that the user needs the smart watch he wears to be tighter in the mobile state and to be looser in the immobile state, for example a rest state, the watchband of the smart watch is adjusted to be tighter when the user is in the mobile state and to be looser when the user is in the immobile state.

In an example, the electronic apparatus further includes a first sensor which is a pressure sensor. The electronic apparatus may be worn on a first location of the user, such as the wrist of the user. In particular, the first detection unit 601 detects a force of the electronic apparatus by the first sensor when the electronic apparatus is worn on the first location; the first analyzing unit 602 determines whether the detected force is within a first predetermined range or not and generates a first determination result; the first command unit 603 generates a first instruction when the first determination result indicates the detected force is within the first predetermined range, and/or the first command unit 603 generates a second instruction when the first determination result indicates the detected force is not within the first predetermined range; correspondingly, the first adjusting unit 604 controls the first body into the first configuration in response to the first instruction, and/or controls the first body into the second configuration in response to the second instruction.

In the above solution, the pressure sensor detects a pressure value between the wrist and the smart watch to acquire the wearing tension by detecting the pressure value. Since the user who wears one smart watch is relatively fixed and his wrist has a relatively fixed size in a certain period, the pressure value of the smart watch at the wrist is recorded in advance by a recording unit (not shown in FIG. 6) of the electronic apparatus when the user feels it is worn tightly, so as to obtain a first predetermined pressure range; and/or the pressure value of the smart watch at the wrist is recorded in advance by the recording unit when the user feels it is worn loosely, so as to obtain a second predetermined pressure range. With reference to the example that only the first predetermined pressure range is recorded and the first configuration corresponds to the loose state of the watchband, when the first detection unit 601 detects the current pressure value by the pressure sensor, the first analyzing unit 602 determines whether the current pressure value detected is in the first predetermined pressure range or not; if yes, it indicates that the smart watch is currently worn too tightly and the first command unit 603 will generate a corresponding instruction to trigger the first adjusting unit 604 to loosen the watchband, otherwise, to trigger the first adjusting unit 604 to tighten the watchband.

In another example, the electronic apparatus further includes a second sensor which is a gravity sensor or an acceleration sensor. In particular, the first detection unit 601 detects a first movement parameter of the electronic apparatus by the second sensor; the first analyzing unit 602 acquires the variation of the first movement parameter within a first predetermined time to determine whether the variation meets a first variation condition or not and to generate a second determination result; the first command unit 603 generates a first instruction when the second determination result indicates the variation meets the first variation condition, and/or, the first command unit 603 generates a second instruction when the second determination result indicates the variation does not meet the first variation condition; correspondingly, the first adjusting unit 604 controls the first body into the first configuration in response to the first instruction, and/or, controls the first body into the second configuration in response to the second instruction.

In the above solution, when the second sensor is the gravity sensor, the first movement parameter is the speed; and when the second sensor is the acceleration sensor, the first movement parameter is the acceleration. The first detection unit 601 detects the speed of the smart watch by the gravity sensor or detects the acceleration of the smart watch by the acceleration sensor. Since the variation of the speed or the acceleration of the user who wears one smart watch and the user himself/herself have certain characteristics within a certain period when the user is in mobile state or in immobile state, the variation range of the speed or acceleration of the smart watch is recorded in advance by the recording unit of the electronic apparatus when the user is in the immobile state within a certain period such as 30 minutes and the recorded variation range is regarded as the first variation condition. With reference to the example that the second sensor is the acceleration sensor and the first configuration corresponds to the loose state of the watchband, the first detection unit 601 detects the acceleration of the smart watch by the acceleration sensor one time per 10 minutes within the 30 minutes and the first analyzing unit 602 calculates the variation rate of the acceleration of the smart watch within the 30 minutes to determine whether the variation rate meets the first variation condition, that is, is in the variation range recorded in advance or not; if yes, it indicates that the user is in the immobile state within the 30 minutes and the first adjusting instruction will be generated by the first command unit 603 to trigger the first adjusting unit 604 to loosen the watchband, otherwise, it indicates that the user is in the mobile state within the 30 minutes and the second adjusting instruction will be generated by the first command unit 603 to trigger the first adjusting unit 604 to tighten the watchband. The method for acquiring the speed by the gravity sensor can be found in the relevant prior art. The details will be omitted herein.

In a further example, the electronic apparatus further includes a third sensor which may be a sensor for detecting the physiological data such as heart rate, blood pressure or pulse, of the user, in particular, may be a resonance sensor. The electronic apparatus may be worn on a first location of the user, such as the wrist of the user. In particular, the first detection unit 601 acquires first data by the third sensor, the first data representing the physiological data of the user; the first analyzing unit 602 determines whether the first data goes beyond a first preset range within a second preset period or not and generates a third determination result; the first command unit 603 generates a first instruction when the third determination result indicates the first data does not go beyond the first preset range within the second preset period, and/or the first command unit 603 generates a second instruction when the third determination result indicates the first data goes beyond the first preset range within the second preset period; correspondingly, the first adjusting unit 604 controls the first body into the first configuration in response to the first instruction, and/or, controls the first body into the second configuration in response to the second instruction.

In the above solution, the first detection unit 601 detects the physiological data such as heart rate, blood pressure or pulse, of the user by the resonance sensor. As the heart rate, blood pressure, pulse of the user who wears one smart watch may change significantly when the user is in mobile state or in immobile state, the variation of the physiological data of the user is recorded in advance by the recording unit of the electronic apparatus when the user is in the immobile state and the recorded variation of the physiological data is regarded as the first preset range. With reference to the example that the resonance sensor detects the blood pressure and the first configuration corresponds to the loose state of the watchband, the first detection unit 601 detects the blood pressure of the user by the resonance sensor. The first analyzing unit 602 determines whether the detected blood pressure goes beyond the first preset range, that is, is in the variation range recorded in advance within a certain period such as 6 minutes or not; if yes, it indicates that the user is in the immobile state and the first adjusting instruction will be generated by the first command unit 603 to trigger the first adjusting unit 604 to loosen the watchband, otherwise, if not, it indicates that the user is in the mobile state and the second adjusting instruction will be generated by the first command unit 603 to trigger the first adjusting unit 604 to tighten the watchband.

Thus, in the first embodiment of the present disclosure, it may determine whether the watchband of the smart watch should be loosened or tightened by means of the analyzing result of the detected first parameter of the smart watch. It can adjust the tension of the wearable apparatus automatically to improve the user's experience, which can exhibit the diversity of function of an electronic apparatus.

With reference to the electronic apparatus described above and shown in FIG. 6, the electronic apparatus further comprises a first device and the first body at least comprises a first deformable body.

The first body is configured to carry out a first operation on the first deformable body by controlling the first device to cause a first deformation of the first deformable body, for example, by means of the first adjusting unit 604 or a component having adjusting function incorporated in the first body. The first body is in the first configuration when the amount of the first deformation meets a first deformation condition.

In an embodiment, the watchband of the smart watch is a soft watchband, for example, is made from rubber or soft leather. The first deformable body may be the gas bag. The soft watchband may include an entire gas bag, or may include a plurality of gas bags. They are not limited herein. The first device may be a gas pump, preferably, may be configured in the body of the smart watch (the part of the smart watch other than the watchband), or may be arranged in the watchband.

As shown in FIG. 4, the watchband is made of an entire gas bag, and includes an inner layer 21, an intermediate layer 22 and an outer layer 23. The inner layer 21 is inflatable and the outer layer 23 may be inflatable or may not be inflatable. The intermediate layer 22 is a hollow layer.

With reference to the example that the first configuration corresponds to the loose state of the watchband (i.e., it determines the user is in the immobile state currently and the watch is worn tightly and needs to be loosened), when the smart watch, in particular the first adjusting unit 604 detects the operation of the user to the first predetermined key, which is used to deflate the watchband, in response to this operation, the gas pump is controlled to perform the deflation operation to the intermediate layer 22. During the deflation, the volume of the intermediate layer 22 is reduced gradually such that the volume of the watchband becomes smaller. As the intermediate layer 22 is deflated continuously, the inner layer 21 reduces the tension to the wrist, i.e., reduces the pressure value of the watchband at the wrist such that the smart watch becomes more loose. The amount of released gas of the intermediate layer 22 is recorded in advance by the recording unit of the electronic apparatus when the wrist of the user feels good; in the course of deflating the intermediate layer 22 of the soft watchband, when the first adjusting unit 604 determines the amount of released gas to reach the recorded value, the gas pump is forbidden to continue deflating the intermediate layer 22 to stop the deflation operation such that the user has a good comfort.

In addition or alternatively, the first adjusting unit 604 is also configured to carry out a second operation on the first deformable body by the first device to cause a second deformation of the first deformable body. When the amount of the second deformation meets a second deformation condition, the first body is in the second configuration. The volume of the first deformable body in the first deformation is less than that in the second deformation.

Herein, as illustrated in FIG. 4, with reference to the example that the second configuration corresponds to the tight state of the watchband (i.e., it determines the user is in the mobile state currently and the watch is worn loosely and needs to be tightened), when the smart watch, in particular the first adjusting unit 604, detects the operation of the user to the second predetermined key, which is used to inflate the watchband, in response to this operation, the gas pump performs the inflation operation to the intermediate layer 22. During the inflation, the volume of the intermediate layer 22 is increased gradually such that the volume of the watchband becomes larger. As the intermediate layer 22 is inflated continuously, the inflatable inner layer 21 expands gradually to press the wrist tightly to increase the pressure value of the watchband at the wrist such that the smart watch becomes tighter. The amount of filling gas of the intermediate layer 22 is recorded in advance by the recording unit of the electronic apparatus when the wrist of the user feels good; in the course of inflating the intermediate layer 22 of the soft watchband, in particular when the first adjusting unit 604 determines the amount of filling gas reaches the recorded value, the gas pump is forbidden to continue inflating the intermediate layer 22 to stop the inflation operation such that the user has a good comfort.

In the above solution, it is explained with reference to the example that the intermediate layer 22 is deflated and inflated by the gas pump to achieve the adjusting of wearing tension of the watch. In addition, the intermediate layer 22 may also carry certain electrolyte which may be heated to cause the expanding of the intermediate layer 22. The electrolyte may be cooled to cause the intermediate layer 22 to contract. That is, the tension of the watchband to the wrist may be increased or reduced by heating or cooling the electrolyte. In particular, with reference to the example that the second configuration corresponds to the tight state of the watchband (i.e., it determines that the user is in the mobile state currently and the watch is worn loosely and needs to be tightened), when the smart watch, in particular, the first adjusting unit 604 detects operation of the user to the third predetermined key, which is used to heat the electrolyte in the intermediate layer 22 of the watchband, in response to the operation, the electrolyte is heated to cause the expanding of the intermediate layer 22. Thus, the pressure value between the watch and the wrist is increased. The heating time of the electrolyte is recorded in advance by the recording unit of the electronic apparatus when the wrist of the user feels good. In the course of heating the electrolyte in the intermediate layer 22, when the first adjusting unit 604 determines the heating time reaches the recorded value, the heating of the electrolyte will be forbidden, i.e., stop the heating such that the user has a good comfort.

As discussed above, in an embodiment of the present disclosure, the object of adjusting the wearing tension of the watch may be achieved by inflating and deflating the intermediate layer by the gas pump and/or by heating and cooling the electrolyte in the intermediate layer. It can adjust the tension of the wearable apparatus automatically to improve the user's experience, which can exhibit the diversity of function of an electronic apparatus.

With reference to the electronic apparatus described above and shown in FIG. 6, the electronic apparatus further includes the second device. The first body at least includes N sub-bodies, where N is a positive integer.

The watchband provided by the present embodiment is a metal watchband. The sub-bodies are sub-watchbands, that is, the watchband includes N sections of sub-bodies. The second device may be a motor, in particular a micro-motor, which may be arranged in each of the sub-watchbands. Each of the sub-watchbands may include a first portion and a second portion. The lengths of the sub-watchbands may be increased or reduced by adjusting the tension of fasteners of the first portion and the second portion so as to increase or reduce the length of the entire watchband and achieve the adjusting of the tension.

When the first body is in the first configuration, the first body has a first length. When the first body is in the second configuration, the first body has a second length. The first length is greater than the second length. That is, the first configuration corresponds to the loose state and the second configuration corresponds to the tight state. The length of the watchband in the loose state is greater than that in the tight state.

The first adjusting unit 604 is configured to generate a first force by the second device, and at least one of the N sub-bodies produces a first displacement in a first predetermined direction under the first force to form the first body having a first length.

In FIG. 5(a), the watchband is a metal watchband, which is formed by N sub-watchbands 51 connected together. In particular, FIGS. 5(b)-5(c) are cross-sectional views of one sub-watchband in different states. In FIGS. 5(b)-5(c), the first portion 510 of each of the sub-watchbands 51 includes a micro-motor 5101 and the second portion 511 includes M fasteners (fastener 1, fastener 2 , . . . , fastener M), where M is a positive integer.

In the embodiment, it is defined as follows: the length of the sub-watchband formed by engaging the first portion 510 with the fastener 1 of the second portion 511 is greater than the length of the sub-watchband formed by engaging the first portion 510 with the fastener 2 of the second portion 511, and the length of the sub-watchband formed by engaging the first portion 510 with the fastener 2 of the second portion 511 is greater than the length of the sub-watchband formed by engaging the first portion 510 with the fastener 3 of the second portion 511, and do on.

Herein, with reference to the example that the first configuration corresponds to the loose state of the watchband (i.e., it determines the user is in the immobile state currently and the watch is worn tightly, for example, the first portion 510 is engaged with the fastener 4 of the second portion 511, and needs to be loosened), when the smart watch, in particular the first adjusting unit 603 detects the operation of the user to a fourth predetermined key which is used to extend the sub-watchband, the micro-motor 5101 produces the first force in response to this operation and under the first force, as shown in FIG. 5(b), the first portion 510 that was previously engaged with the fastener 4 of the second portion 511 is engaged with the fastener 1 such that the engagement of the first portion 510 and the second portion 511 becomes looser and the sub-watchband becomes longer in the x axis to lengthen the entire watchband, that is, it obtains the longer watchband to achieve the adjusting of the wearing tension.

In addition or alternatively, the first adjusting unit 604 is also configured to generate a second force by the second device, and at least one of the N sub-bodies produces a second displacement in a second predetermined direction under the second force to form the second body having a second length.

Herein, with reference to the example that it determines the user is in the mobile state currently and the watch is worn loosely, for example, the first portion 510 is engaged with the fastener 1 of the second portion 511, and needs to be tightened (i.e., the second configuration corresponds to the tight state of the watchband), when the smart watch, in particular the first adjusting unit 603 detects the operation of the user to a fifth predetermined key which is used to shorten the sub-watchband, the micro-motor 5101 produces the second force in response to this operation and under the second force, as shown in FIG. 5(c), the first portion 510 that was previously engaged with the fastener 1 of the second portion 511 is engaged with the fastener 4 such that the engagement of the first portion 510 and the second portion 511 becomes tighter and the sub-watchband becomes shorter in the x axis to shorten the entire watchband, that is, it obtains the shorter watchband to achieve the adjusting of the wearing tension.

In the above solution, the variation of the length of the sub-watchband is achieved by varying the fastener of the second portion engaged with the first portion. Further, the first portion and the second portion may also be connected by springs or elastic connecting portions (such as elastic strings) to lengthen or shorten the sub-watchband by increasing or reducing the elasticity of these connecting portions, thereby achieving the adjusting of wearing tension.

As discussed above, in the embodiment of the present disclosure, the sub-watchband may be lengthened or shortened to lengthen or shorten the entire watchband. It can adjust the tension of the wearable apparatus automatically to improve the user's experience, which can exhibit the diversity of function of an electronic apparatus.

It should be noted that, in the first embodiment of the present disclosure, the first detection unit, the first analyzing unit and the first adjusting unit are incorporated in the control unit, or these units may be separate units, or may be an entire device integrated together as long as their function can be achieved. They are not limited herein.

### Second Embodiment

In the prior art, the configuration of the wearable apparatus is fixed, or may be adjusted manually by the user, instead of being adjusting automatically to meet the different requirements of the user to the configuration in different states.

Regarding the above issues in the prior art, the second embodiment of the present disclosure provides a wearable apparatus which is smartly adjustable. The wearable apparatus includes a first portion and a second portion. The first portion and the second portion are combined to form the whole frame (or the whole shape of structure) of the wearable apparatus. The first portion is a deformable structure and may be deformed after receiving a first control instruction such that the shape of the wearable apparatus and/or the force required for deformation of the wearable apparatus is changed. The second portion is different from the first portion.

The wearable apparatus includes a control unit. In the example, the control unit is a controller configured to generate the first control instruction from the received parameter information.

In the wearable apparatus provided by the second embodiment, the controller adjusts the shape of the wearable apparatus on the basis of the received parameter information to meet the different requirements of the user to the configurations in different states. And the parameter information in the embodiment of the present disclosure may be certain information of the user himself, or may be certain environmental parameter information. When the controller adjusts the wearable apparatus on the basis of these parameters, it may meet different requirements of the user to the configuration in different states.

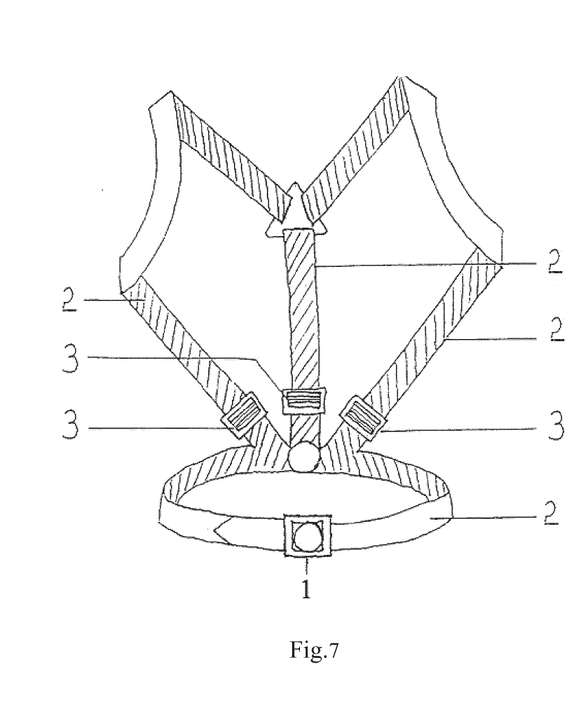

As shown in FIG. 7, the second embodiment of the present disclosure provides a wearable apparatus. The second embodiment will be described in more details with reference to the drawings below.

The wearable apparatus includes a first portion (i.e., the first body) 3 and a second portion (i.e., the second body) 2. The first portion 3 and the second portion 2 are combined to form the whole frame (or the whole shape of structure) of the wearable apparatus. The first portion 3 is a deformable structure and may be deformed after receiving a first control instruction such that the shape of the wearable apparatus and/or the force required for deformation of the wearable apparatus is changed. The second portion is different from the first portion.

In the second embodiment of the present disclosure, in order to adjust the posture and/or body type of the user, the second portion may be formed from deformable materials, or may be formed from non-deformable materials.

The controller 1 is configured to generate the first control instruction from the received parameter information.

FIG. 7 shows schematically a structure of the technical solution of the second embodiment of the present disclosure applied in a posture correction belt. However, the technical solution provided by the embodiment of the present disclosure is not only limited to use in the posture correction belt, but also in any wearable apparatus, in particular the wearable apparatus having an adjusting function.

In order to control the first portion 3 to carry out the relative operation on the basis of the control instruction transmitted by the controller 1, the controller 1 and the first portion 3 may be connected with each other by wires or wireless. When the first portion 3 and the controller 1 are connected with each other by wires, the wires for connection are made from flexible materials so as to improve the comfort without influencing the outer shape of the wearable apparatus.

In the prior art, although an article such as the posture correction belt may adjust the posture of the user, the force applied by the posture correction belt to the user often goes beyond the normal range of the force that the user can burden. If it is used for a long time, it may cause obstruction of the blood flow of the user. Thus, in order to improve the therapeutic effects, in the technical solution provided by the second embodiment of the present disclosure, the shape of the posture correction belt may be adjusted in real-time according to the time. Therefore, in the embodiment of the present disclosure:

the controller 1 is further configured to do timekeeping after the control instruction is transmitted; if the duration for which timekeeping is done is greater than a time threshold, an alarm information for prompting the user to adjust the shape of the wearable apparatus will be generated.

The technical solutions provided by the second embodiment of the present disclosure may be applied in the posture correction belt, however, it is not limited only to the application in the posture correction belt, and also can be used in adjusting type underclothes for women. As illustrated in FIG. 7, when it is used in the posture correction belt, the first portion 3 includes:

a first assembly made from deformable materials (a structure as shown in FIG. 7, the first assembly may be the part connecting the second portion 2 with the force transmission assembly) and a force transmission assembly, and the force transmission assembly changes the force applied to the first assembly to change the shape of the first assembly. After the force transmission assembly receives the first control instruction, the force applied to the first assembly is adjusted on the basis of the first control instruction to adjust the shape of the first portion.

In the embodiment, the force transmission assembly may be a reel or a stretchable member. In the specific environment of application, the force transmission assembly can adjust the shape of the entire wearable apparatus by changing factors such as its shape or length.

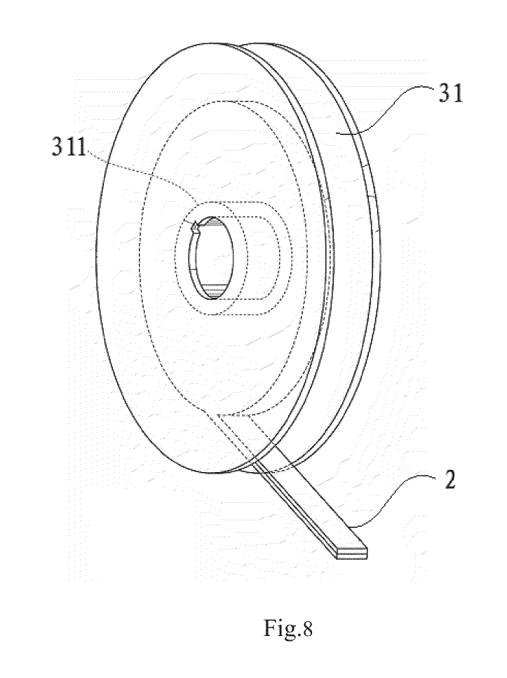

In an example, when the force transmission assembly is a controllable reel (as shown in FIG. 8), a control portion 311 in the controllable reel 31 may carry out the corresponding operation on the basis of the first control instruction to control the reel 31 to rotate about its axis center so as to change the force applied by the posture correction belt to the user, and thereby adjust the shape of the posture correction belt.

In addition, in order to achieve deformable effects, the first portion 3 may also be another structure. In view of the advantages of lightness and high comfort of an inflatable structure, the first portion 3 may includes:

a gas deflation and aeration assembly and a gas pressure assembly, and after the gas pressure assembly receives the first control instruction, the amount of gas entering the gas deflation and aeration assembly is adjusted on the basis of the first control instruction to adjust the shape of the first portion.

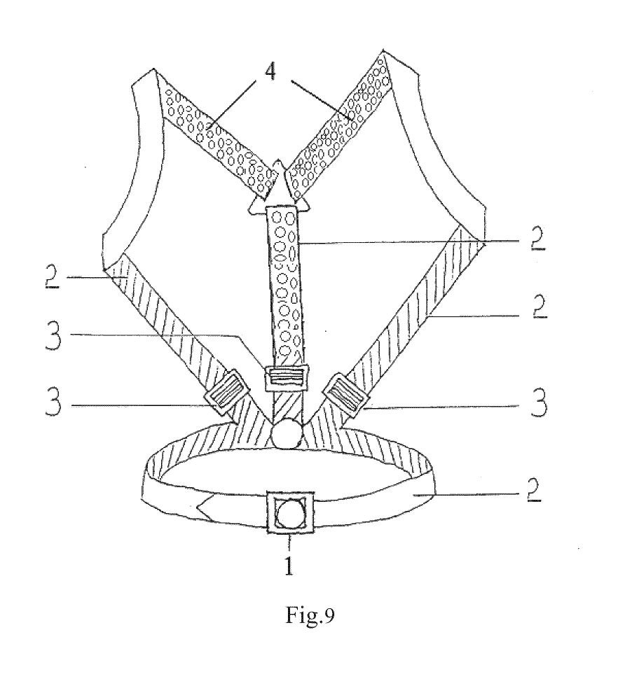

As illustrated in FIG. 9, in the second embodiment of the present disclosure, as the article for posture correction may apply a relative large force to the human body and an excessive pressure will cause uncomforting of the user when the first portion and the second portion of the wearable apparatus are made from hard materials, a gas cushion buffer assembly 4 may be provided in the second embodiment of the present disclosure, in particular, the gas cushion buffer assembly 4 may be provided on the sides of the first portion and the second portion contacting with the human body.

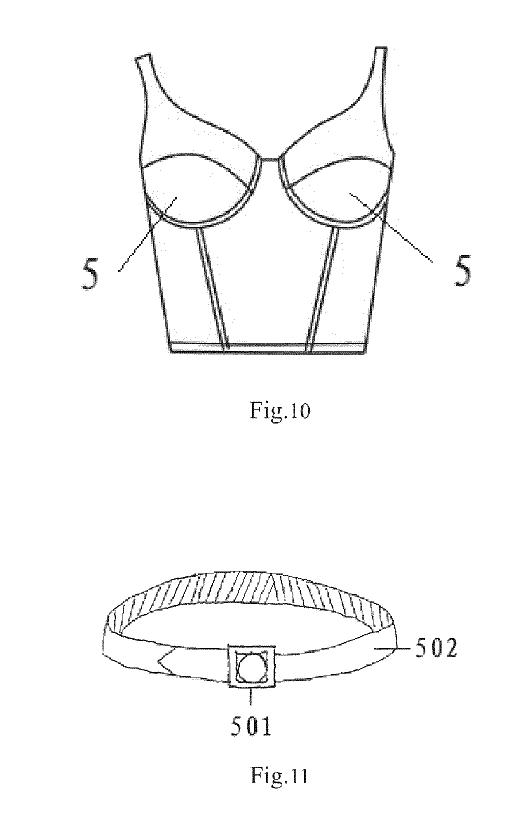

As illustrated in FIG. 10, when the technical solutions provided by the second embodiment of the present disclosure are applied in the adjusting type underclothes for women and the first portion 3 is the above gas deflation and aeration assembly and gas pressure assembly, the gas deflation and aeration assembly may be arranged on the chest position of the adjusting type underclothes for women (see position indicated by reference numeral 5 in FIG. 10), and the appearance of the deflation and inflation may be adjusted so as to achieve the effects of adjusting shapes.

As illustrated in FIG. 11, the technical solutions provided by the second embodiment of the present disclosure may also be applied in a waistband in the wearable apparatus. In particular, the waistband includes the first portion 501 and the second portion 502. The first portion 501 is made from deformable material or is a deformation adjusting structure. In the embodiment, the second portion 502 may be made from flexible materials. In the embodiment shown in FIG. 11, the first portion 501 may be a reel. When the first portion acts, the length of the second portion may be adjusted to change the size of the diameter of the waistband.

In the embodiment shown in FIG. 11, the first portion is arranged at the position of the fastener of waistband. In the specific application, the first portion 501 may be arranged on any position of the waistband as long as the size of the diameter of the waistband can be adjusted.

Because the waistband will have different requirements on deformation when the human walks, stands, sits or lies, the user may control the deformable materials of the waistband on the basis of the current posture, such that the entire waistband becomes into the desired configuration. Further, the controller adjusts the size of maximum stretch of the waistband or the restriction force of the waistband automatically according to the custom of the user, for example the time just before eating or the pressure of the stomach. In other words, the controller controls the waistband automatically such that the same deformation of the waistband needs to be applied by different forces.

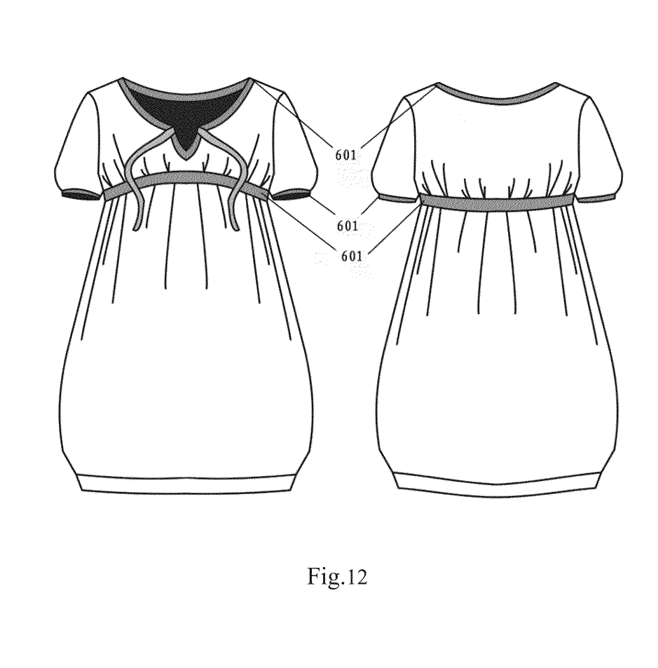

As shown in FIG. 12, when the wearable apparatus according to the second embodiment of the present disclosure is clothing, the first portion 601 which is deformable or may have a controllable deformation may be arranged at, for example, the waist portion, shoulder portion, arm portion or collar portion of the clothing. In the specific application, the first portion 601 may be arranged on all of a plurality of positions, as shown in FIG. 12, or may be arranged on one of the plurality of positions. After one or several certain positions are selected for the first portion, other portions will be the second portion. In an example, the first portion may be a controllable reel. According to the example shown in FIG. 12, if the first portion is arranged at the waist portion and the controller controls the controllable reel of the first portion, the waist portion of the clothing may be adjusted by tightening up the controllable reel to cause wrinkles or by tightening down the controllable reel to remove wrinkles.

With reference to the above embodiments of waistband and the clothing, other conventional apparels may have the effects of adjusting the shapes in the same manner as the technical solutions provided by the embodiment of the present disclosure.

Further, in order that the first control instruction generated by the controller 1 can be fitted to the requirements of the user and the current environmental conditions, in the wearable apparatus provided by the second embodiment of the present disclosure, the first control instruction is generated by the sensor configured to detect the environmental space parameters in a predetermined range. The sensor includes one or more of a pressure sensor, a temperature sensor and a humidity sensor and any combination thereof.

The controller is further configured to receive the environmental space parameter and to generate the first control instruction from the received environmental space parameter.

Alternatively, it may also be configured to generate the first control instruction according to habits and custom of the user.

The controller is further configured to generate the first control instruction on the basis of a predetermined first rule which is generated according to habits and custom of the user who uses the wearable apparatus.

In another alternative example, the first control instruction is generated on the basis of a control command transmitted by a cell phone or other mobile terminals by the user.

The wearable apparatus further includes a wireless transceiver module which is configured to receive the control command transmitted from the terminals such as cell phone and to transmit the control command to the controller to generate the first control instruction.

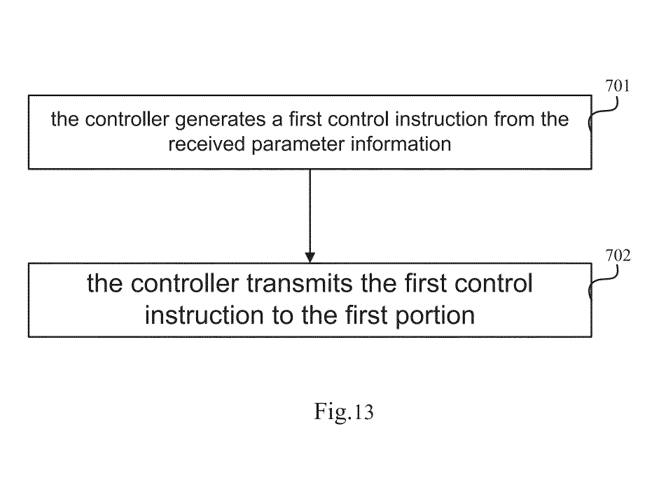

As illustrated in FIG. 13, the second embodiment of the present disclosure also provides a data process method applied in a controller. The controller is fitted to the wearable apparatus in any one of the above embodiments as long as the whole frame (or the whole shape of structure) of the wearable apparatus is formed by combining the first portion with the second portion. The first portion may be a deformable structure and the second portion is different from the first portion. When the controller is used to control the wearable apparatus to deform, the method comprises:

Step 701: generating a first control instruction by the controller from the received parameter information.

The modes receiving the parameter information by the controller may include:

detecting an environmental space parameter by a sensor in a predetermined range, the environmental space parameter being regarded as the parameter information, the sensor comprising one or more of a pressure sensor, a temperature sensor and a humidity sensor and any combination thereof;

generating the first control instruction on the basis of a predetermined first rule which is generated according to habits and custom of the user who uses the wearable apparatus.

generating the first control instruction by receiving the control instruction transmitted by a receiving terminal of a wireless transceiver module.

Step 702: transmitting the first control instruction by the controller to the first portion, wherein it causes the first portion to deform after the first control instruction is received such that the shape of the wearable apparatus changes and/or the force required for producing deformation of the wearable apparatus changes.

The above one or more technical solutions in the embodiments of the present application at least have the following technical effects:

In the wearable apparatus provided by the second embodiment of the present disclosure, the controller adjusts the shape of the wearable apparatus according to the received parameter information to avoid the incorrect posture caused by manual improper adjustment. And in the embodiment of the present disclosure, the parameter information may be certain information of the user himself, or may be certain environmental parameter information. When these parameters are adjusted by the controller, the posture of the user may be adjusted while improving the comfort of the user in use.

In the above embodiment of the present application, it should be noted that the disclosed apparatus and method may be implemented by other means. The above embodiments of apparatus are only exemplary. For example, division of the units is only a division of logical functions. In practice, other division modes are applicable, for example, a plurality of units or assemblies may be combined or may be integrated in another system or some features may be omitted or may not be implemented. In addition, the coupling between the shown or discussed components or direct coupling or communication connections may be implemented by indirect couplings or communication connections of some interfaces, apparatuses or units, may be electrical, mechanical or in other forms.

The above units described as separate components may be or may not be separate physically. The component shown as units may be physical units or may not be physical units. That is, it may be located at one position, or may be distributed on a plurality of network units. It may select part or all of the units as requirements in practice to achieve the technical solutions of the embodiment.

Further, all of functional units in the embodiments of the present disclosure may be integrated in one process unit, or the respective units are regarded as one unit separately, or two or more units may be integrated in one unit. The above integrated unit may not only be implemented in form of hardware, but also may be implemented in form of hardware and software functional unit.

The skilled person in the art can understand: all or part of the steps in the above embodiments of the method may be implemented by hardware associated with the program instructions. The above program may be stored in a computer readable storage medium. When the program is executed, the steps comprising the above embodiments of the method are executed. The above storage medium includes: all kinds of media which can store the program code, such as mobile storage devices, read-only memory(ROM), magnetic discs or optical discs.

Some parts of the following embodiments are implemented in form of algorithms which include operation for the data stored in the computer memory. The algorithms substantially mean the self consistent sequence for operation causing the desired results. These operations typically need or is associated with physical manipulation or physical amounts. Generally, but not necessary, these amounts take in form of electrical signals or magnetic signals. These signals can be stored, transmitted, emerged, compared and manipulated in other manners. It has proved that it is often convenient to call these signals as bits, values, elements, symbols, signs, terms, numbers, typically for the sake of use.

However, it should be noted that all of these and similar terms are associated with suitable physical amounts, and are only labels for convenience in use of these amounts. The description for the terms such as “process” or “calculate” or “determine” or “display” used throughout the description may mean the action and process executed by the data process system or similar electronic device, unless it is described apparently otherwise. The action and process manipulate data represented by physical (electronic) amounts in the registers and memories of the computer and convert them into other data represented in form of physical amounts similarly in the memories or registers or other such information storage, transmission or display devices) of the system.

The present disclosure may relate to an apparatus for executing one or more of the operations in the present application. The apparatus may be constructed specially for a desired object or may comprise a general computer which is selectively activated or reconfigured by computer programs stored in the computer. Such computer program may be stored in a machine (such as computer) readable medium or in any type of media which are suitable for storing electronic instructions and coupled into the bus respectively. The computer readable medium includes, but not limited to, any types of discs (including floppy discs, optical discs, CD-ROM and magnetic-optical discs), read-only memory(ROM), random access memory (RAM), erasable programmable ROM (EPROM), electrically erasable programmable ROM (EEPROM), flash memory, magnetic cards or optical cards.

The machine readable medium includes any mechanism for storing or transmitting information in machine (for example computer) readable form. For example, it includes read-only memory(ROM), random access memory (RAM), magnet storage medium, optical storage medium, flash memory device, signals that are progressed in electrical, optical, acoustic or the other forms (such as carriers, infrared signals, digital signals and the like).

The method according to the present disclosure is not limited to the above embodiments, and other embodiments conceived by the skilled person in the art from the technical solutions of the present disclosure also belong to the scope of the present disclosure.

The above embodiments are only those of the present disclosure by way of examples. The scope of the present disclosure is not limited to this. Any modifications and variations may be envisaged by the skilled person in the art apparently from these embodiments without departing from the technical scope of the disclosure should fall within the scope of the present disclosure. Therefore, the scope of the present disclosure is defined in the appended claims and their equivalents.

What is claimed is:

1. A wearable apparatus, comprising:
a first body having different configurations; and
a control unit configured to acquire a first parameter and generate a control instruction based on the first parameter to adjust the first body to one of said different configurations.

2. The wearable apparatus as claimed in claim 1, wherein the control unit comprises:
a first detection unit configured to detect the first parameter;
a first analyzing unit configured to analyze the first parameter to acquire a first analyzing result; and
a first command unit configured to generate the control instruction based on the first analyzing result to adjust the first body to one of said different configurations.

3. The wearable apparatus as claimed in claim 2, wherein the control unit further comprises a sensor configured to acquire the first parameter, the sensor being one of a pressure sensor, a gravity sensor, an acceleration sensor, and a sensor for detecting physiological data of a human body.

4. The wearable apparatus as claimed in claim 2, wherein the first body comprises at least a first device and a first deformable body, the first device being operable to deform the first deformable body.

5. The wearable apparatus as claimed in claim 4, wherein the wearable apparatus is a smart watch, the first device being a gas pump and the first deformable body being a gasbag in a watchband of the smart watch, and a tension of the watchband is adjustable by the gas pump.

6. The wearable apparatus as claimed in claim 5, wherein the gasbag comprises an electrolyte and the tension of the watchband is further adjustable by either heating or cooling the electrolyte, or a combination of both.

7. The wearable apparatus as claimed in claim 2, wherein the wearable apparatus is a smart watch, the first body comprising a plurality of sub-watchbands constituting a watchband of the smart watch, each of the sub-watchbands comprising a micro-motor and a plurality of fasteners, and a length of each of the sub-watchbands is adjustable depending on an engagement between a respective one of the micro-motors and a corresponding one of the fasteners.

8. The wearable apparatus as claimed in claim 7, wherein the first body further comprises a first adjusting unit;
the first adjusting unit is operable to cause the micro-motor to produce a first force, and at least one of the sub-watchbands is operable to produce a first displacement under the first force to adjust the first body to have a first length; or
the first adjusting unit is operable to cause the micro-motor to produce a second force, and at least one of the sub-watchbands is operable to produce a second displacement under the second force to adjust the first body to have a second length, wherein the second length is different from the first length.

9. The wearable apparatus as claimed in claim 7, wherein the micro-motor is connected with the fasteners by an elastic member.

10. The wearable apparatus as claimed in claim 1, further comprising a second body, wherein the first body and the second body are combined to form the whole frame of the wearable apparatus.

11. The wearable apparatus as claimed in claim 10, wherein the first body comprises a first assembly formed by a deformable material and a force transmission assembly, and the force transmission assembly is operable to change a force applied to the first assembly to adjust the first assembly to one of said different configurations based on the acquired first parameter.

12. The wearable apparatus as claimed in claim 11, wherein the force transmission assembly is a controllable reel.

13. The wearable apparatus as claimed in claim 10, wherein the first body comprises a gas deflation and aeration assembly and a gas pressure assembly, and the first body being operable to adjust an amount of gas entering the gas deflation and aeration assembly based on the control instruction to adjust the first body to one of said different configurations based on the acquired first parameter.

14. The wearable apparatus as claimed in claim 10, further comprising a gas cushion buffer assembly for contacting with a human body.

15. The wearable apparatus as claimed in claim 10, further comprising:
a sensor configured to detect an environmental space parameter in a predetermined range, the sensor being one or more of a pressure sensor, a temperature sensor, a humidity sensor, and
wherein the control unit is further configured to receive the environmental space parameter and to generate the control instruction based on the received environmental space parameter.

16. The wearable apparatus as claimed in claim 10, wherein the control unit is further configured to generate the control instruction according to habits or customs of the user who uses the wearable apparatus.

17. The wearable apparatus as claimed in claim 10, wherein the control unit is configured to generate the control instruction based on a control command transmitted by the user.

18. The wearable apparatus as claimed in claim 10, wherein the control unit is further configured to do timekeeping after the control instruction is transmitted; and if the duration for which timekeeping is done is greater than a time threshold, the control unit is operable to generate an alarm information for prompting the user to adjust the first body to one of said different configurations.

19. A data processing method configured to process data in a wearable apparatus which comprises a first body having different configurations to be worn by a user and a control unit configured to generate a control instruction based on a received first parameter to adjust the first body, the method comprising:
detecting the first parameter as the wearable device is worn by the user;
analyzing the first parameter; and
adjusting the first body to one of said different configurations based on the analyzed first parameter.

20. A data processing method for processing data in a wearable apparatus which comprises a first body and a control unit, the first body having different configurations; the method comprising:
generating a control instruction by the control unit based on a received parameter information;
transmitting the control instruction to the first body; and
adjusting the first body to one of the different configurations based on the control instruction.
